(19) United States
(12) Patent Application Publication
(10) Pub. No.: US 2010/0030252 A1
(43) Pub. Date: Feb. 4, 2010

(54) NASAL DILATOR

(76) Inventor: Keith Stewart, East Lothian (GB)

Correspondence Address: DYKAS, SHAVER & NIPPER, LLP, P.O. BOX 877, BOISE, ID, 83701-0877, US

(21) Appl. No.: 12/295,773

(22) PCT Filed: Apr. 3, 2007
(86) PCT No.: PCT/GB07/01298

§ 371 Date: Mar. 16, 2009

(30) Foreign Application Priority Data
Apr. 3, 2006 (GB) 0606669.0

(51) Int. Cl. A61M 29/00

(52) U.S. Cl. 606/199

(57) ABSTRACT

A nasal dilator is described. The nasal dilator comprises at least two opposing limbs (1) that are biased apart and that are configured so as to be capable of being inserted simultaneously into a single nostril. In one embodiment, the dilator comprises two limbs and the limbs are configured such that once both limbs are inserted they may perform an outward dilation of the nostril at the soft tissue surrounding the ostium internum.

### FIELD OF THE INVENTION

The present invention relates to a nasal dilator, in particular but not exclusively, to nasal dilators that can improve inhalation and exhalation through the nose and thereby alleviate problems associated with chronic or temporary nasal congestion. Additionally, the invention relates to methods of inserting the nasal dilator into the nose and the treatment of chronic or temporary nasal congestion.

### BACKGROUND TO THE INVENTION

Mucus is a viscous colloid made by goblet cells in the mucous membranes of the nose. Mucus contains antiseptic enzymes (such as lysozyme), immunoglobulins, mucins and inorganic salts suspended in water. In the respiratory system, it catches foreign matter and tries to prevent it from entering the body. Mucus is normally secreted in only one nostril at a time, and the nostril secreting the mucus changes about every 4 to 6 hours. Although the secretion of mucus has important physiological functions, abnormally increased mucus production in the respiratory tract is problematic and is a symptom of many common diseases, such as the common cold. The presence of mucus in the nose and throat is normal, but increased quantities can impede comfortable breathing.

Nasal congestion is the blockage of the nasal passages and is usually due to excessive secretion of mucus. As mucus is usually secreted in one nostril at a time, it is normal to have only a single nostril blocked at any one time. Nasal congestion has many causes and can range from a mild annoyance to a life-threatening condition. Nasal congestion can interfere with the ears, hearing, and speech development. Significant congestion may interfere with sleep, cause snoring, can be associated with sleep apnea, can result in insufficient oxygen levels and hypoxia, as well as right-sided heart failure. A blocked nose can also causes mild facial and head pain.

The anatomical structure of the human nose contributes to the difficulties experienced when breathing. Specifically, the lower internal portion of each nostril, known as the vestibule, typically tapers inward to a narrowed area, known as the ostium internum, which defines the opening of the nasal air passage defined by the isthmus nasi (running from 1.65 to 2.65 cm from the nostril, and is typically about 0.3 cm in diameter). It is this narrowed portion of the nose that can become blocked with mucus. The ostium internum (located about 1 to 1.5 cm from the nostril) is an orifice whose pear-shaped format, visualized under anterior rhinoscopy, is laterally limited by the inferior border of lateral superior cartilage, medially to the nasal septum and inferiorly to the floor of the nasal cavity. Chemical decongestants, such as pseudoephidrine and oxymetazoline, are commonly used to clear nasal decongestion. These chemicals work by reducing inflammation of the nasal cavity and acting as vasoconstrictors. However, where possible, it is not desirable to use such chemical interventions as they can be associated with harmful side affects.

Cage or stent like devices have also been used to combat nasal decongestion, see for example U.S. Pat. No. 4,414,977. Such devices work by acting as fixed volume dilators and are placed inside the nostril. However, these dilators are not capable of expanding the breadth of the nasal cavity passed the fixed volume, are uncomfortable to wear, and difficult to retrieve after use.

Nasal dilators are known that include a pair of outwardly biased opposing limbs that are designed for inserting one limb into each nostril (see, for example, British Patent Publication No. 2 330 079). Such devices expand the outermost fleshy portion of the vestibule of the nose which can be uncomfortable and may result in a transient or even permanent stretching of that tissue, which can be undesirable for cosmetic reasons. Additionally, as both nostrils are being dilated at the same time, each nostril cannot be optimally dilated. There is a finite amount of tissue forming the nostril, which is capable of a finite amount of stretch, and so if both nostrils are being stretched in two opposing directions optimum dilation of each nostril is not possible.

### SUMMARY OF THE INVENTION

It is therefore an object of the invention to provide a nasal dilator that is capable of alleviating breathing problems associated with nasal congestion, but that does not possess the problems associated with the prior art, as discussed above.

According to a first aspect of the present invention there is provided a nasal dilator comprising at least two opposing limbs that are biased apart and that are configured so as to be capable of being inserted simultaneously into a single nostril.

It has surprisingly been found that inserting such a dilator into the nostril of the nasal passage that is unblocked can provide the optimum dilation of the nasal passage that cannot be achieved by a dilator that, for example, dilates both nasal passages simultaneously. Dilation of the soft tissue of the vestibule does not provide any benefit so the nasal dilator is preferably configured such that once both limbs are inserted they may perform an outward dilation of the nostril at the soft tissue surrounding the ostium internum.

The applicant has found that one of the simplest ways in which to construct such a dilator is by connecting the opposing limbs by a resiliently deformable bridge. Such a construction may be created by moulding (e.g. injection moulding) the device so as to leave the mould with the at least two opposing limbs connected by a resiliently deformable bridge configuration.

Preferably, the dilator is arranged such that the deformable bridge is adjacent an end of each limb.

Preferably, the limbs extend from the bridge in substantially the same direction.

Most preferably, the limbs diverge as they extend away from the bridge. Divergence of the limbs ensures the optimum dilation of the nasal passage without extending the nostril entrance.

Alternatively, the dilator may be formed from a length of material comprising a notch, around which the length of material may be bent so as to form the resiliently deformable bridge. In one embodiment, the length of material extending from the notch defining the limbs. Such simple constructions have the advantage of being cheap and easy to manufacture.

In order to configure the dilator so as to be inserted easily into the nose it is preferred that the thickness of the limbs taper from the thickest point at the bridge. Preferably the diameter of each limb at its thickest point is less than 0.3, 0.25, 0.2 or 0.1 cm. All limbs are preferably of equal length, and so maintain equal pressure on the nasal cavity from each limb and so prevent any torsional forces. The applicant has found that in order for the limbs to extend a sufficient distance into the nasal cavity so as to dilate the tissue around the ostium internum each limb is preferably more than 1, 1.5, 2 or 2.5 cm in length.

For comfort of the user it is also preferred that the nasal dilator further comprises a cushioned portion provided on each limb. The cushioned portions may be provided at or towards the end of each limb that is furthest removed from the bridge. It has been found that cushioned portions that mould or are shaped to the contours of the nasal cavity are most comfortable, for example concaved pads. Concaved pads are preferred as they match the contours of the nasal cavity and provide only limited obstruction to the airway. The cushioned portions may be provided directly onto the limbs or may provided on a stalk that laterally extends from the end of the limb that is furthest removed from the bridge. The stalk may be off reduced thickness to the rest of the limb so that the outer surface of the limb and the cushioned portion can be continuous. The cushioned portion maybe sleeves. The cushioned portions may be bonded to the stalks or limbs or maybe moulded onto the stalks or limbs. The stalks or limbs may include a projecting portion to retain the cushioned pads thereon.

The applicant has found that it may be desirable to include a feature of the invention that prevents the device from being inhaled or pushed too far into the nasal cavity. Thus, the dilator may include at least one stop means associated with at least one limb and configured such that the dilator cannot be inserted into the nasal cavity passed the stop means.

The stop means preferably extends laterally from at least one limb. The stop means may alternatively be formed by at least one laterally extending kink in the at least one limb. In such a confirmation the bend or kink along the otherwise substantially straight limb can block the insertion of the dilator passed the kink in the limb. Two kinks may be provided on one limb with a finger grip portion defined by the portion of the limb provided between the kinks. Alternatively, the stop means may also act as a finger grip portion. In the interests of cheap and efficient manufacture the stop means may be formed integrally with at least one limb. In order to provide the required blocking, the stop means preferably extends more than 1 cm from the limb, or the kink extends more than 1 cm from the line of the limb. It is preferred that each limb extends more than 1.5, 2 or 2.5 cm from the stop means to the end of each limb that is furthest removed from the bridge. Preferably the same stop means is provided in all limbs. An alternative or additional safety feature that could be included in the present invention is an elbow bend in at least one limb. Such a bend in the limb prevents the insertion of the dilator passed the point in the limb of the bend. Preferably the elbow defines an angle in the limb of substantially 90° and/or the same elbow bend is provided in all limbs.

The applicant has found that in order to spread the outward pressure from the dilator and optimise the dilation of the nasal cavity the dilator may be provided with a plurality of limbs, for example, 2, 3, 4, 5 or more limbs. The limbs are preferably equally biased away from each other.

Embodiments of the present invention may be configured for use during the day, when a device extending from the nose is not desired, for cosmetic reasons. Thus, the limbs and resiliently deformable bridge may be configured, when in use, so as to be entirely housed within the nasal cavity. Such configurations may be achieved in a number of ways, however, the inventor has found the following two embodiments to be particularly effective. The resiliently deformable bridge may take the form of a horse shoe with each limb extending in a perpendicular manner therefrom. The horse shoe shaped bridge may be shaped so as to mirror the shape of the internal nasal cavity. Alternatively, the resiliently deformable bridge may be formed from a first and second resiliently deformable u-bend that are connected by a third resiliently deformable u-bend, wherein the third resiliently deformable u-bend is inverted in relation to the first and second deformable u-bends, i.e. the resiliently deformable bridge takes a “M” shape.

In a further preferred embodiment of the present invention, the nasal dilator may comprise a passageway through which air can flow. In this way, even if the dilator is inserted into a blocked nasal cavity the passageway may pass through the blockage and the limbs help to expand the area for the passageway to pass through. The passageway may be associated with the nasal dilator in a number of ways, for example, the passageway can be associated with a limb from the bridge or attached directly to the bridge. The passageway may be a tubular member. Such a tubular member can be moveably retained on a limb. In order to improve the ease in which such a dilator may be inserted the passageway can be rotatably mounted and/or made of a flexible material and/or a mesh. As the passageway may need to pass further into the nasal cavity than the opposed limbs, possibly even into the isthmus nasi, the limb associated with the passageway may extend further from the bridge than the other limbs. In order to prevent the passageway from becoming blocked, it preferably includes a mesh provided at its end furthest removed from the bridge.

The dilator should be made of a light material so that it may be easily worn in the nose without causing any discomfort. The nasal dilator is preferably less than 1, 0.5 or 0.25 g in weight. The nasal dilator may be made from synthetic plastics, such as nylon, polyethylene, polypropylene, or any combination thereof. Such a dilator can be cheaply made by integral moulding.

In a preferred embodiment, the nasal dilator comprises acetal, most preferably medical grade acetal. Acetal is advantageous because it has good shape memory and nasal dilators of specific tensions can be manufactured. Acetal is also very resistant to bending fatigue.

In an alternative embodiment, the nasal dilator comprises polycarbonate. Polycarbonate lends itself to being adjusted by the user to a preferred tension.

In order to prevent the need to wash the dilator or risk the transfer of infection the dilator is preferably disposable.

In a second aspect of the present invention, there is provided a method of inserting a nasal dilator comprising the steps of urging together opposing limbs of a dilator that comprises at least two biased apart limbs so as to reduce the profile of the dilator, passing all limbs of the reduced profile dilator in through a single nostril, and releasing the limbs such that they spring apart providing an outward dilation of the single nostril.

The dilator is preferably passed through the nostril until they may perform an outward dilation of the nostril at the soft tissue surrounding the ostium internum.

Preferably, there are two limbs.

Preferably, the step of passing the limbs in through a single nostril comprises inserting the limbs such that a plane through the limbs is substantially parallel to the nasal septum.

Preferably, the step of passing the limbs in through a single nostril comprises rotating the nasal dilator such that the plane through the limbs is substantially perpendicular to the septum.

Preferably, once the limbs are released the limbs outwardly dilate a side of the nostril away from the septum.

The method may include a first step of locating the unblocked nostril for inserting the dilator into. This can simply be achieved by the user closing one nostril and exhaling through the open nostril, if exhalation is difficult then the open nostril is congested. Alternatively, the method may involve inserting one dilator into each nostril.

The method may be used to maintain an open airway and may therefore make it easier for the user to achieve the state of sleep. The method may be used to treat nasal congestion.

Preferably the dilator used in the second aspect of the present invention is any one of the dilators described in the first aspect of the present invention.

It will be understood that any of the features of the first aspect may be applicable to the second aspect and are not repeated for brevity.

### BRIEF DESCRIPTION OF THE DRAWINGS

The present invention will now be described, by way of example, with reference to the accompanying drawings, in which:

FIG. 1 shows an embodiment of the present invention with two limbs.

FIG. 2 shows an embodiment of the present invention with three limbs.

FIG. 3 shows an embodiment of the present invention with two non-linear limbs.

FIG. 4 shows an embodiment of the present invention with three non-linear limbs.

FIG. 5 shows an embodiment of the present invention with an elbow bend.

FIG. 6 shows an embodiment of the present invention with a horse shoe shaped resiliently deformable bridge.

FIG. 7 shows an embodiment of the present invention with an “M” shaped resiliently deformable bridge.

FIG. 8 shows an embodiment of the present invention with a passageway through which air may flow.

FIG. 9 shows an embodiment of the present invention with combined spars and grip portions.

FIG. 10 shows an embodiment of the present invention with a notched bridge.

FIG. 11 shows an embodiment of the present invention with a reduced profile.

### DETAILED DESCRIPTION OF THE DRAWINGS

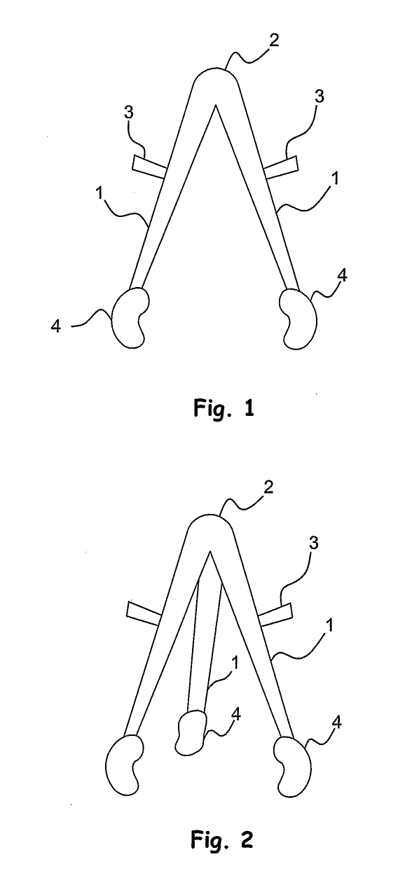

The nasal dilator of the present invention, as illustrated in FIG. 1, comprises a pair of opposing limbs (1) that are connected by a resiliently deformable bridge (2). A spar (3) extends laterally from each limb (1) and concave pads (4) are provided at the end of each limb (1) that is furthest removed from the bridge (2). In use, the spars (3) engage with the outer portion of the nostril during insertion of the dilator into the nostril, thereby preventing insertion passed the point on the limb (1) from where the spars (3) extend.

A further embodiment of the nasal dilator of the present invention can be seen in FIG. 2. This embodiment is similar to the embodiment shown in FIG. 1, but has three limbs (1). Each limb (1) is equally biased, and so spaced, apart.

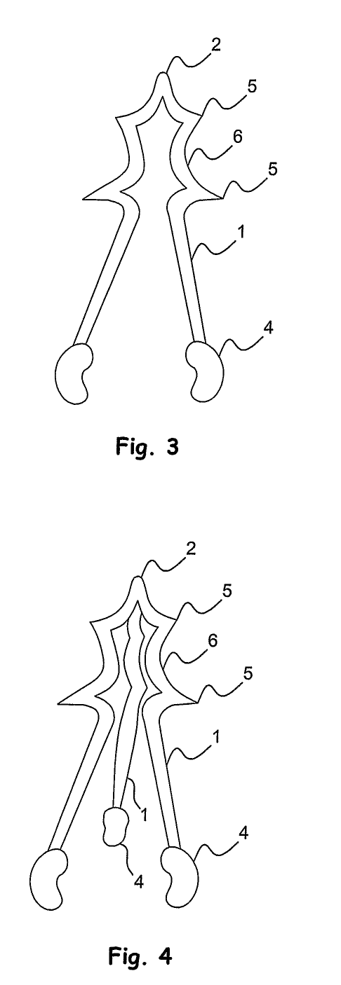

A further embodiment of the nasal dilator of the present invention can be seen in FIG. 3. This embodiment is similar to the embodiment shown in FIG. 1, but instead of including a spar (3), each limb includes two kinks (5) at the portion of each limb that is closest to the bridge (2). Between these kinks (5) is defined a grip portion (6) that is sized so as to be suitable to accommodate a finger tip. In use, the user may grip the dilator between two fingers, each finger resting in the grip portion (6). The kinks (5) engage with the outer portion of the nostril during insertion of the dilator into the nostril, thereby preventing insertion passed the point on the limb (1) from where the kink (5) that is furthest removed from the bridge (2) is formed.

A further embodiment of the nasal dilator of the present invention can be seen in FIG. 4. This embodiment is similar to the embodiment shown in FIG. 3, but has three limbs (1). Each limb (1) is equally biased, and so spaced, apart.

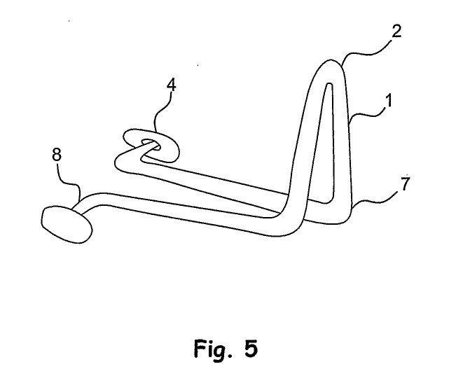

A further embodiment of the nasal dilator of the present invention can be seen in FIG. 5. In this embodiment the limbs (1) include identical 90 degree bends (7) in the portion of each limb (1) towards the bridge (2). The limbs (1) include protrusions (8) that laterally extend from the end of each limb (1) that is furthest removed from the bridge (2). The protrusions (8) laterally extend away from the protrusion (8) on the opposing limb (1). Concave pads are provided at the end of each lateral protrusion. In use, the portion of the limb (1) that extends from the 90 degree bend (7) to the bridge (2) engages with the outer portion of the nostril during insertion of the dilator into the nostril, thereby preventing insertion passed the 90 degree bend (7). In use the outward bias of the limbs (1) urges the concave pads (4) towards the soft tissue within the nasal cavity that surrounds the ostium internum.

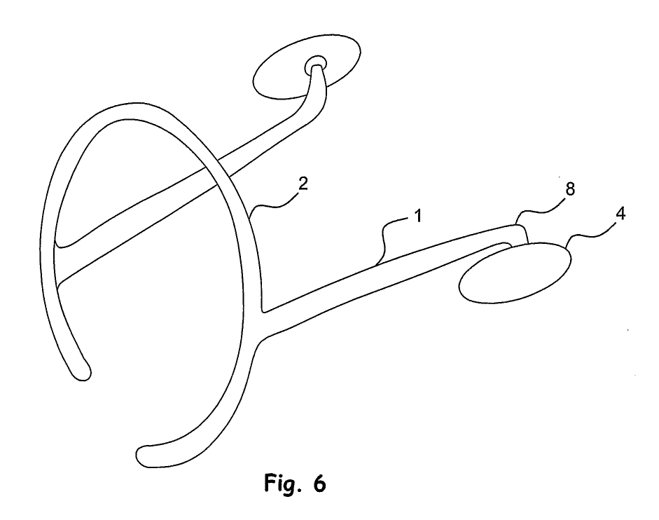

A further embodiment of the nasal dilator of the present invention can be seen in FIG. 6. In this embodiment the deformable bridge (2) is not formed from a u-bend, like earlier embodiments, but is instead provided in the shape of a horse shoe. A limb (1) extends in a perpendicular manner from the horse shoe shaped bridge (2) at each of the portions provided towards the free ends of the horse shoe (2). As the horse shoe bridge (2) is resiliently deformable, in use, it may be squeezed between two fingers, thereby narrowing the profile of the horse shoe (2) and bringing the limbs (1) closer together. In such a squeezed configuration, the dilator may easily pass entirely into the nasal cavity. When the pressure applied to the bridge (2) is removed, once the dilator has been inserted into the nasal cavity, the bridge (2) is able to return to its original shape which conforms to the shape of the interior of the nasal cavity and the outward bias of the limbs (1) urges the concave pads (4) towards the soft tissue within the nasal cavity that surrounds the ostium internum.

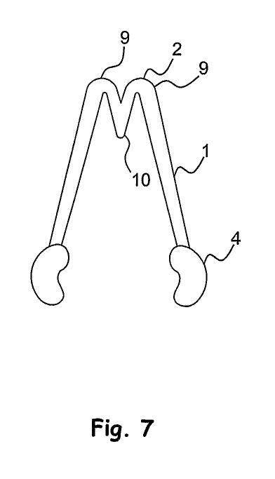

A further embodiment of the nasal dilator of the present invention can be seen in FIG. 7. In this embodiment the deformable bridge (2) is formed from a first and second resiliently deformable u-bend (9) that are connected by a third resiliently deformable u-bend (10), which is inverted in relation to the first and second deformable bridge (10). Thus, the bridge (2) takes the form of an “M”.

In the previous two embodiments of the present invention, the configurations permit the dilator to be entirely housed within the nasal cavity.

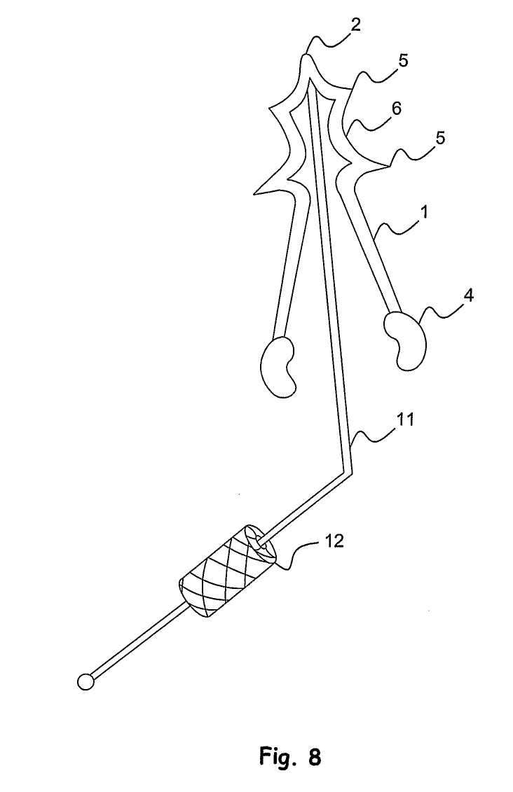

A further embodiment of the nasal dilator of the present invention can be seen in FIG. 8. This embodiment is similar to the embodiment illustrated in FIG. 3, but includes a longer limb (11) that extends further than the other two limbs (1). This longer limb (11) includes a tube (12) rotatably mounted around a portion of the longer limb (11). The tube (12) is made from a mesh. In use, the dilator can be inserted into the nostril in much the same manner as described for FIG. 3 above, however, the third limb (11) is inserted passed the ostium internum and into the isthmus nasi. When inserted, the tube is positioned in the isthmus nasi and forces the walls of this cavity apart thereby defining a passageway through which air can travel. This is assisted by the outwardly biased limbs (1). The longer limb (11) and the tube (12) are flexible, enabling them to be configured to the shape of the nasal passageway. The tube (12) can move longitudinally and rotate on the third limb (11) so as to be positioned in at the appropriate position along the longer limb (11).

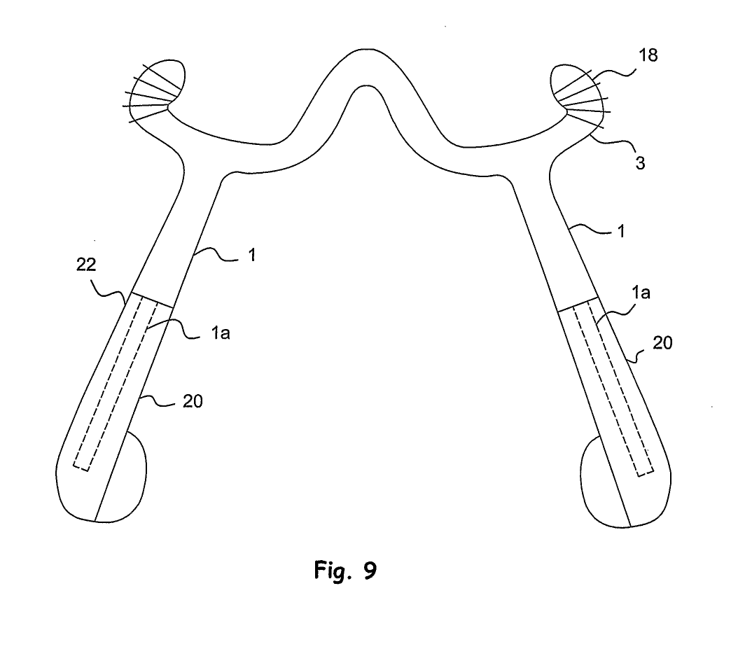

FIG. 9 shows a still further embodiment of the present invention with combined spars (3) and grip portions (18). In this embodiment each limb (1) defines a reduced lower limb section (1a, shown in broken outline) over which cushioned sleeves (20) can be positioned. The cushioned sleeves (20) are moulded out of a soft elastomer and are bonded to the reduced limb sections (1a) to provide a continuous outer limb surface (22). The cushioned sleeves (20) define a rounded end (24) to assist in easing the dilator into the nasal cavity with minimum discomfort to the wearer.

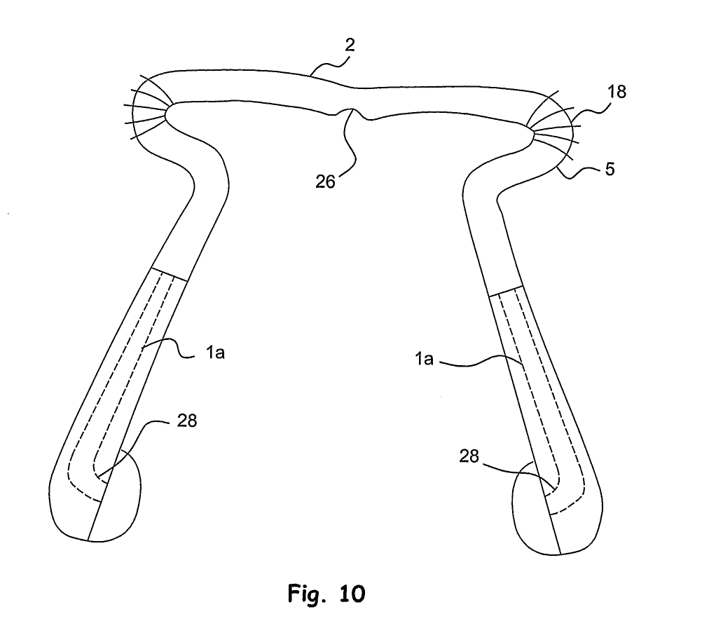

FIG. 10 shows an embodiment of the present invention with a notched bridge (2). The bridge (2) of the dilator bends around a notch (26) located at the midpoint of the bridge (2). The bridge (2) also includes opposing kinks (5) which prevent over insertion over the dilator into the nasal cavity. As can be seen from FIG. 10, the kinks (5) also define grip portions (18). In this embodiment, the lower ends of the reduced limb portions (1a) define projections (28). Rather than being moulded separately, cushioned sleeves (20) are moulded over the lower limb portions (1a), the projections (28) being provided to prevent the cushioned sleeves (20) from slipping off.

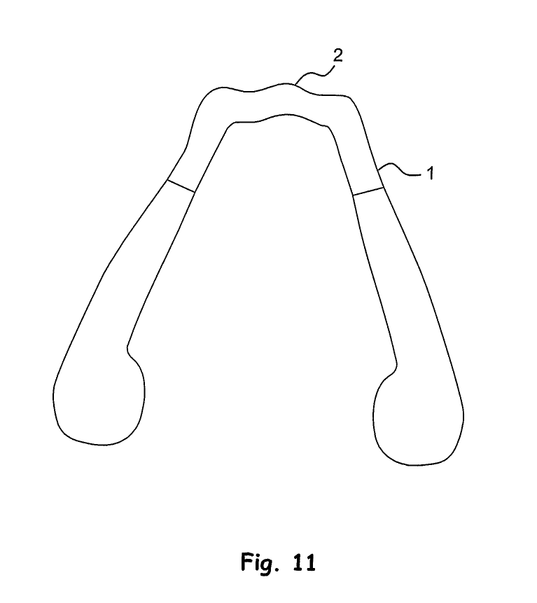

FIG. 11 shows an embodiment of the present invention with a reduced profile. In this embodiment, the bridge (2) is fairly flat so that the dilator can be inserted fully into the nose and worn more discreetly.

When using the above devices, the user will first determine which one of the nasal passageways that is blocked by physically closing one nostril by pinching it with ones fingers and exhaling through the remaining open nasal passageway. If it is difficult to exhale through the open passageway then this passageway is congested. If exhalation is easy then this is the nostril in which the dilator is to be inserted in the above mentioned manner.

The present invention has been described above purely by way of example. It should be understood that modifications in detail may be made within the scope of the invention.

1) A nasal dilator comprising at least two opposing limbs that are biased apart and that are configured so as to be capable of being inserted simultaneously into a single nostril.

2) A nasal dilator as claimed in claim 1, that is configured such that once said limbs are inserted they may perform an outward dilation of the nostril at the soft tissue surrounding the ostium internum.

3) A nasal dilator as claimed in claim 1, wherein the opposing limbs are connected by a resiliently deformable bridge.

4) A nasal dilator as claimed in claim 3, wherein the dilator is moulded so as to form at least two opposing limbs connected by the deformable bridge

5) A nasal dilator as claimed in claim 3, formed from a length of material comprising a notch, around which the length of material may be bent so as to form the resiliently deformable bridge with the lengths of material extending from the notch defining the limbs.

6) A nasal dilator as claimed in claim 3, wherein the thickness of the limbs taper from the thickest point at the bridge.

7) A nasal dilator as claimed in claim 1, further comprising a cushioned portion provided on each limb.

8) A nasal dilator as claimed in claim 7, wherein the cushioned portion is provided at or towards the end of each limb that are furthermost removed from the bridge.

9) A nasal dilator as claimed in claim 7, wherein the cushioned portion takes the form of a concaved pad.

10) A nasal dilator as claimed in claim 7, wherein the cushioned portion is provided on a stalk that laterally extends from the end of the limb that is furthest removed from the bridge.

11) A nasal dilator as claimed in claim 7, the preceding claims, further comprising at least one stop means associated with at least one limb and configured such that the dilator cannot be inserted into the nasal cavity passed the stop means.

12) A nasal dilator as claimed in claim 11, wherein at least one stop means extends laterally from at least one limb.

13) A nasal dilator as claimed in claim 11, wherein at least one stop means is formed integrally with the at least one limb.

14) A nasal dilator as claimed in claim 13, wherein the stop means if formed form a laterally extending kink in at least one limb.

15) A nasal dilator as claimed in any of claim 12, wherein the stop means extends more than 1 cm from the limb.

16) A nasal dilator as claimed in claim 1, wherein each limb is more than 1, 1.5, 2 or 2.5 cm in length.

17) A nasal dilator as claimed in claim 11, wherein each limb extends more than 1.5, 2 or 2.5 cm from the stop means to the end of each limb that is further removed from the bridge.

18) A nasal dilator as claimed in claim 1, wherein all limbs are of equal length.

19) A nasal dilator as claimed in claim 1, wherein each limb is less than 0.3, 0.25, 0.2 or 0.1 cm thick.

20) A nasal dilator as claimed in claim 1, wherein at least one limb includes an elbow bend.

21) A nasal dilator as claimed in claim 20, wherein the elbow defines an angle in the limb of substantially 90°.

22) A nasal dilator as claimed in claim 20, wherein the same elbow bend is provided in all limbs.

23) A nasal dilator as claimed in claim 1, comprising 2, 3, 4, 5 or more limbs.

24) A nasal dilator as claimed in claim 23, wherein all limbs is equally biased away from each limb.

25) A nasal dilator as claimed in claim 1, wherein the limbs and resiliently deformable bridge are configured, when in use, so as to be entirely housed within the nasal cavity.

26) A nasal dilator as claimed in claim 25, wherein the resiliently deformable bridge takes the form of a horse shoe with each limb extending in a perpendicular manner therefrom.

27) A nasal dilator as claimed in claim 25, wherein the resiliently deformable bridge is formed from a first and second resiliently deformable u-bend that are connected by a third resiliently deformable u-bend, wherein the third resiliently deformable bridge is inverted in relation to the first and second deformable bridge.

28) A nasal dilator as claimed in claim 1, further comprising a passageway through which air can flow.

29) A nasal dilator as claimed in claim 27, wherein the passageway is associated with a limb.

30) A nasal dilator as claimed in claim 28, wherein the passageway is defined by a tubular member.

31) A nasal dilator as claimed in claim 30, wherein the tubular member is moveably retained on a limb.

32) A nasal dilator as claimed in claim 30, wherein the tubular member is formed from a mesh.

33) A nasal dilator as claimed in claim 29, wherein the limb associated with the passageway extends further into the nasal cavity than the other limbs.

34) A nasal dilator as claimed in claim 28, wherein the passageway is attached to the bridge.

35) A nasal dilator as claimed in claim 31, wherein the passageway is rotatably mounted.

36) A nasal dilator as claimed in any of claim 28, wherein the passageway and/or limb associated with the passageway is made of a flexible material.

37) A nasal dilator as claimed in claim 1, wherein the dilator is disposable.

38) A nasal dilator as claimed in claim 1 that is less than 1 g in weight.

39) A nasal dilator as claimed in claim 1 that is less than 0.5 g in weight.

40) A nasal dilator as claimed in claim 1 that is less than 0.25 g in weight.

41) A nasal dilator as claimed in claim 1 that is made of a synthetic plastics.

42) A nasal dilator as claimed in claim 35 that is made from nylon, polyethylene, polypropylene, or any combination thereof.

43) A nasal dilator as claimed in claim 1, wherein the at least one stop means comprises a grip portion.

44) A nasal dilator as claimed in claim 7, wherein the stalk is of reduced thickness to the rest of the limb so that the outer surface of the limb and the cushioned portion can be continuous

45) A nasal dilator as claimed in claim 7, wherein the cushioned portion is a sleeve.

46) A nasal dilator as claimed in claim 7, wherein the cushioned portions are bonded to the stalks or limbs.

47) A nasal dilator as claimed in claim 7, wherein the cushioned portions are moulded onto the stalks or limbs.

48) A nasal dilator as claimed in claim 7, wherein the stalks include a projecting portion to retain the cushioned pads

49) A method of inserting a nasal dilator into a single nostril comprising the steps of urging together opposing limbs of a dilator that comprises at least two biased apart limbs so as to reduce the profile of the dilator, passing all limbs of the reduced profile dilator in through a single nostril, and releasing the limbs such that they spring apart providing an outward dilation of the single nostril.

50) A method as claimed in claim 49, further including a first step of locating the unblocked nostril for inserting the dilator into.

51) A method as claimed in claim 49, wherein there are two limbs.

52) A method as claimed in claim 51, wherein the step of passing the limbs in through a single nostril comprises inserting the limbs such that a plane through the limbs is substantially parallel to the septum.

53) A method as claimed in claim 52, wherein the step of passing the limbs in through a single nostril comprises rotating the nasal dilator such that the plane through the limbs is substantially perpendicular to the septum.

54) A method as claimed in claim 49, wherein once the limbs are released the limbs outwardly dilate a side of the nostril away from the septum.

55) (canceled)

56) (canceled)
